(19) United States
(12) Patent Application Publication
(10) Pub. No.: US 2012/0107419 A1
(43) Pub. Date: May 3, 2012

(54) ORAL RINSE FORMULATION AND METHOD OF USE

(76) Inventor: Raj Sadasivan, Sibley, MO (US)

(21) Appl. No.: 12/914,272

(22) Filed: Oct. 28, 2010

(51) Int. Cl. A61K 33/14; A61P 43/00; A61P 17/02

(52) U.S. Cl. 424/679; 424/680

(57) ABSTRACT

A formulation and method of use for neutralizing the acidity in the mouth of a patient. The method comprises taking into the mouth an aqueous solution comprising a water soluble inorganic salt consisting of sodium and/or potassium chloride, a sodium phosphate buffer consisting of sodium monophosphate and/or sodium diphosphate and glycerin in a pH-balanced solution, maintaining the aqueous solution in the mouth for a period of time sufficient to reduce the acidity in the mouth, and thereafter spitting out the aqueous solution. The amount of sodium/potassium chloride present in the aqueous solution may be from about 0.45% to about 2.0% by weight per unit volume. The amount of sodium monophosphate in the aqueous solution may be from about 0.005% to about 0.02% by weight per unit volume, for example. The amount of sodium diphosphate in the aqueous solution may be from about 0.005% to about 0.02% by weight per unit volume. The amount of glycerin in the aqueous solution may be about 0.0005% to about 0.002% by weight per unit volume. The pH of the aqueous solution may be from 6.5 to 7.5. The period of time to maintain the aqueous solution in the mouth may be from about 30 seconds to two minutes.

### FIELD

The present invention relates to formulations and their method of use in the maintenance and improvement of oral health and, more particularly, formulations and their method of use for patients with chemotherapy and/or radiation therapy induced oral mucositis, mouth sores, mouth pain, oral ulcers, sore gums, dry mouth and the like.

### BACKGROUND

Oral mucositis is a painful inflammation and ulceration of the mucous membranes of the mouth and is commonly an adverse effect of chemotherapy and/or radiation therapy, particularly for patients undergoing head and neck radiation therapy. Cancer patients undergoing chemotherapy typically become symptomatic after one week after beginning treatment while mucositis associated with radiotherapy typically appears approximately two weeks after beginning treatment. The mucosal cells of the mouth are injured in reaction to chemotherapy and/or radiotherapy resulting in the development of oral lesions, which may become red, inflamed and ulcerated. The pain associated with oral mucositis can be severe resulting in the patient experiencing trouble speaking, eating or opening his or her mouth. Sores or ulcerations may become infected acting as a portal of entry for oral flora that may cause septicemia, especially in immuno-suppressed patients. Development of oral mucositis may result in alterations or changes to the chemotherapy dose such that the prognosis may be adversely affected.

Typical treatment of oral mucositis may include oral hygiene by rinsing the mouth with water or salt water and avoidance of citrus fruits, alcohol and spicy foods. Other medicinal treatments may be used such as chlorhexidine gluconate and viscons Lidocaine for relief of pain, for example. However, these medicinal treatments should not be swallowed and primarily treat the symptoms, not the problem.

### SUMMARY

The present invention includes a formulation and method of use for neutralizing the acidity in the mouth of a patient. The method comprises taking into the mouth an aqueous solution comprising a water soluble inorganic salt consisting of sodium and/or potassium chloride, a sodium phosphate buffer consisting of sodium monophosphate and/or sodium diphosphate and glycerin in a pH-balanced solution, maintaining the aqueous solution in the mouth for a period of time sufficient to reduce the acidity in the mouth, and thereafter spitting out the aqueous solution. The amount of sodium/potassium chloride present in the aqueous solution may be from about 0.45% to about 2.0% by weight per unit volume, for example. The amount of sodium monophosphate in the aqueous solution may be from about 0.005% to about 0.02% by weight per unit volume, for example. The amount of sodium diphosphate in the aqueous solution may be from about 0.005% to about 0.02% by weight per unit volume, for example. The amount of glycerin in the aqueous solution may be about 0.0005% to about 0.002% by weight per unit volume, for example. The period of time to maintain the aqueous solution in the mouth may be from about 30 seconds to almost two minutes, for example.

It is anticipated that additional components may include coloring and flavoring additions.

### DETAILED DESCRIPTION

The desired resulting preferred formulation is a mixture of a balanced salt solution with a phosphate buffer. The formulation is a physiologically buffered solution to allow mucous membranes to heal. The formulation includes at least one water soluble inorganic salt consisting of sodium and/or potassium chloride, a sodium phosphate buffer consisting of sodium monophosphate and/or sodium diphosphate and glycerin in concentration hereafter indicated. As an oral mouth rinse for individuals with chemotherapy and/or radiation therapy induced oral mucositis, mouth sores, mouth pain, oral ulcers, more gums and dry mouth, the formulation is a pH-balanced solution which promotes the healing process. The pH of the solution is in the range of approximately 6.5 to 7.5, and preferably 6.8 to 7.4.

In one embodiment, the formulation is prepared and packaged in a liquid form as an aqueous solution in the solution concentration for its end use. More specifically, the sodium/potassium chloride is employed in a solution concentration of between approximately 0.45% and 2% weight per unit volume, preferably between approximately 0.6% and 1.2%. The sodium phosphate buffer is employed in a solution concentration of between approximately 0.005% and 0.02% weight per unit volume, preferably between approximately 0.008% and 0.012%. The sodium phosphate buffer may include a combination of sodium monophosphate and sodium diphosphate in concentration between 0.005% and 0.02% weight per unit volume. The glycerin is employed in a solution concentration of between approximately 0.0005% and 0.002% weight per unit volume, preferably between approximately 0.0008% and 0.0012%.

The following, are representative examples of preferred formulations by dissolving 10 grams of each formulation in 1097.7 milliliter quantities of water:

| | | |
|---|---|---|
| | | Percent |
| | | Concentration (gram |
| Component | Weight | per 100 milliliter) |

Formulation A

| | | | | |
|---|---|---|---|---|
| Sodium chloride | 9.88 | gram | 0.9 | gram |
| Sodium monophosphate | 0.055 | gram | 0.005 | gram |
| Sodium diphosphate | 0.055 | gram | 0.005 | gram |
| Glycerin | 0.01 | gram | 0.001 | gram |
| Water | 1097.7 | milliliters | 100 | milliliters |

Formulation B

| | | | | |
|---|---|---|---|---|
| Sodium chloride | 4.94 | gram | 0.45 | gram |
| Potassium chloride | 4.94 | gram | 0.45 | gram |
| Sodium monophosphate | 0.055 | gram | 0.005 | gram |
| Sodium diphosphate | 0.055 | gram | 0.005 | gram |
| Glycerin | 0.01 | gram | 0.001 | gram |
| Water | 1097.7 | milliliters | 100 | milliliters |

Formulation C

| | | | | |
|---|---|---|---|---|
| Potassium chloride | 9.88 | gram | 0.9 | gram |
| Sodium monophosphate | 0.055 | gram | 0.005 | gram |
| Sodium diphosphate | 0.055 | gram | 0.005 | gram |
| Glycerin | 0.01 | gram | 0.001 | gram |
| Water | 1097.7 | milliliters | 100 | milliliters |

Formulation D

| | | | | |
|---|---|---|---|---|
| Sodium chloride | 7.41 | gram | 0.675 | gram |
| Potassium chloride | 2.47 | gram | 0.225 | gram |
| Sodium monophosphate | 0.055 | gram | 0.005 | gram |
| Sodium diphosphate | 0.055 | gram | 0.005 | gram |
| Glycerin | 0.01 | gram | 0.001 | gram |
| Water | 1097.7 | milliliters | 100 | milliliters |

Formulation E

| | | | | |
|---|---|---|---|---|
| Sodium chloride | 2.47 | gram | 0.225 | gram |
| Potassium chloride | 7.41 | gram | 0.675 | gram |
| Sodium monophosphate | 0.055 | gram | 0.005 | gram |
| Sodium diphosphate | 0.055 | gram | 0.005 | gram |
| Glycerin | 0.01 | gram | 0.001 | gram |
| Water | 1097.7 | milliliters | 100 | milliliters |

Formulation F

| | | | | |
|---|---|---|---|---|
| Sodium chloride | 9.88 | gram | 0.9 | gram |
| Sodium monophosphate | 0.079 | gram | 0.007 | gram |
| Sodium diphosphate | 0.1299 | gram | 0.013 | gram |
| Glycerin | 0.01 | gram | 0.001 | gram |
| Water | 1097.7 | milliliters | 100 | milliliters |

Formulation G

| | | | | |
|---|---|---|---|---|
| Sodium chloride | 9.88 | gram | 0.9 | gram |
| Sodium monophosphate | 0.0761 | gram | 0.0075 | gram |
| Sodium diphosphate | 0.1389 | gram | 0.013 | gram |
| Glycerin | 0.01 | gram | 0.001 | gram |
| Water | 1097.7 | milliliters | 100 | milliliters |

Formulation H

| | | | | |
|---|---|---|---|---|
| Sodium chloride | 9.88 | gram | 0.9 | gram |
| Sodium monophosphate | 0.0736 | gram | 0.0075 | gram |
| Sodium diphosphate | 0.1471 | gram | 0.015 | gram |
| Glycerin | 0.01 | gram | 0.001 | gram |
| Water | 1097.7 | milliliters | 100 | milliliters |

Formulation I

| | | | | |
|---|---|---|---|---|
| Sodium chloride | 9.88 | gram | 0.9 | gram |
| Sodium monophosphate | 0.0713 | gram | 0.007 | gram |
| Sodium diphosphate | 0.1542 | gram | 0.015 | gram |
| Glycerin | 0.01 | gram | 0.001 | gram |
| Water | 1097.7 | milliliters | 100 | milliliters |

It is contemplated that the formulation in this embodiment is preferably prepared in the concentration preferred for its end use. More specifically, the salt is employed in a solution concentration of between 0.45% and 2.0% weight per unit volume and preferably between approximately 0.6% and 1.2%. The sodium phosphate buffer is employed in a solution concentration of between 0.005% and 0.02% weight per unit volume and preferably between approximately 0.008% and 0.012%. The glycerin is employed in a solution concentration of between 0.0005% and 0.002% and preferably between approximately 0.0008% and 0.0012%. The above formulations A-I are representative of this embodiment, but liquid formulations according to this embodiment would preferably be produced on a significantly larger scale. Alternatively, it is contemplated that the liquid formulation of this embodiment may be prepared and packaged in a concentrated form for shipping to a location to be diluted or which may be diluted in small amounts when used as an oral rinse. The diluted or concentrated formulations should preferably be packaged in appropriate containers suitable for shipping and handling the liquids, and sealed to prevent contamination. A conventional coloring agent such as FD&C Blue No. 2 may be added to achieve a final concentration of 0.0002% as well as a flavoring agent as desired, for example. Other coloring agents may be added as desired.

The formulation may be administered as a 10 cc to 20 cc oral rinse and preferably a 15 cc (½ ounce) oral rinse to rinse the mouth of a patient for 30 seconds to two minutes and preferably between 45 seconds and 90 seconds. Preferably the solution should be spit out; however, the solution is not toxic. Optimal benefit may be achieved when the solution is used to rinse the mouth two to 10 times daily and preferably three to six times daily. Preferably the solution is used after meals, snacks, chemotherapy and radiation treatments for one to four weeks. Regular use of the formulation may significantly reduce mouth pain, irritation, ulcers, burning sensations, sore gums, swelling and/or redness, for example. Additionally, regular use of the formulation may reduce difficulty in drinking liquids, chewing and swallowing foods, for example.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as set forth in the claims.

Having thus described the invention, what is claimed as new and desired to be secured by Letters Patent is as follows:

1. A method for treatment of chemotherapy and/or radiation therapy induced oral mucositis, mouth sores, mouth pain, oral ulcers and/or sore gums by neutralizing the acidity in the mouth comprising rinsing the oral cavity with an aqueous solution of:
at least one water-soluble inorganic salt selected from the group consisting of sodium chloride and potassium chloride in a total concentration in the range of approximately 0.45% to 2.0% by weight per unit volume,
at least one water-soluble sodium phosphate buffer selected from the group consisting of sodium monophosphate and sodium diphosphate in a total concentration in the range of approximately 0.005% to 0.02% by weight per unit volume,
and glycerin in a concentration in the range of approximately 0.0005% to 0.002% by weight per unit volume, for a period of time sufficient to neutralize the acidity in the mouth.

2. The method of claim 1 wherein said inorganic salt is present in said aqueous solution in a total concentration in the range of approximately 0.6% to 1.2% by weight per unit volume.

3. The method of claim 1 wherein said sodium chloride is present in said aqueous solution in a total concentration in the range of approximately 0.45% to 2.0% by weight per unit volume.

4. The method of claim 1 wherein said potassium chloride is present in said aqueous solution in a total concentration in the range of approximately 0.45% to 2.0% by weight per unit volume.

5. The method of claim 1 wherein said sodium monophosphate is present in said aqueous solution in a total concentration in the range of approximately 0.0005% to 0.02% by weight per unit volume.

6. The method of claim 1 wherein said sodium diphosphate is present in said aqueous solution in a total concentration in the range of approximately 0.0005% to 0.02% by weight per unit volume.

7. A method of neutralizing the acidity in the mouth comprising:
a. taking into the mouth an aqueous solution comprising an inorganic salt, a sodium phosphate buffer and glycerin in an amount effective to neutralize said acidity in the mouth;
b. maintaining said aqueous solution in the mouth for a period of time to neutralize said acidity in the mouth; and
c. thereafter spitting out said aqueous solution whereby the acidity in the mouth is neutralized.

8. The method of claim 7 wherein said inorganic salt is in said aqueous solution in an amount from about 0.45% to 2.0% by weight per unit volume.

9. The method of claim 7 wherein said inorganic salt is selected from the group consisting of sodium chloride and potassium chloride.

10. The method of claim 9 wherein said inorganic salt is in said aqueous solution in an amount from about 0.6% to 1.2% by weight per unit volume.

11. The method of claim 7 wherein said sodium phosphate buffer in said aqueous solution is an amount from about 0.0005% to 0.02% by weight per unit volume.

12. The method of claim 7 wherein said sodium phosphate buffer is selected from the group consisting of sodium monophosphate and sodium diphosphate.

13. The method of claim 12 wherein said sodium phosphate buffer in said aqueous solution is an amount from about 0.008% to 0.012% by weight per unit volume.

14. The method of claim 7 wherein said glycerin in said aqueous solution is an amount from about 0.0005% to 0.002% by weight per unit volume.

15. The method of claim 7 wherein said period of time is in the range of approximately 30 seconds to two minutes.

16. The method of claim 7 wherein said aqueous solution is balanced.

17. The method of claim 16 wherein said pH is in a range of approximately 6.8 to 7.4.

18. The method of claim 7 wherein said amount of said aqueous solution is in the range of approximately 10 cc to 20 cc.

19. The method of claim 7 further comprising a coloring agent in said aqueous solution.

20. The method of claim 19 wherein said coloring agent is in an amount of about 0.0002%.

21. The method of claim 20 wherein said coloring agent is FD&C Blue No. 2.

22. The method of claim 7 further comprising a flavoring agent in said aqueous solution.

23. An aqueous solution formulation for treatment of chemotherapy and/or radiation induced oral mucositis, mouth sores, mouth pain, oral ulcers and/or sore gums and the like by neutralizing the acidity in the mouth, said formulation comprising:
a water-soluble inorganic salt,
a water-soluble sodium phosphate buffer,
glycerin, and
water.

24. The aqueous solution formulation of claim 23 wherein said water-soluble inorganic salt is selected from the group consisting of sodium chloride and potassium chloride.

25. The aqueous solution formulation of claim 24 wherein said water-soluble inorganic salt is in a total concentration in the range of approximately 0.45% to 2.0% by weight per unit volume.

26. The aqueous solution formulation of claim 25 wherein said water-soluble inorganic salt is a mixture of sodium chloride and potassium chloride in generally the same concentrations by weight per unit volume.

27. The aqueous solution formulation of claim 23 wherein said water-soluble sodium phosphate buffer is selected from the group consisting of sodium monophosphate and sodium diphosphate.

28. The aqueous solution formulation of claim 27 wherein said water-soluble sodium phosphate buffer is in a total concentration in the range of approximately 0.005% and 0.02% by weight per unit volume.

29. The aqueous solution formulation of claim 28 wherein said water-soluble sodium phosphate buffer is a mixture of sodium monophosphate and sodium diphosphate in generally the same concentrations by weight per unit volume.

30. The aqueous solution formulation of claim 23 wherein said glycerin is in a total concentration the range of approximately 0.0005% to 0.002% by weight per unit volume.

31. The aqueous solution formulation of claim 23 further comprising a conventional coloring agent.

32. The aqueous solution formulation of claim 23 further comprising a conventional flavoring agent.

33. The aqueous solution formulation of claim 23 wherein said solution is pH balanced.

34. The aqueous solution formulation of claim 23 wherein said solution has a pH in the range of approximately 6.5 to 7.5.

35. The aqueous solution formulation of claim 23 wherein said solution has a pH in the range of approximately 6.8 to 7.4.
